SEQUENCE LISTING (8 sequences)

SEQ ID NO: 1 - protein, 14 aa, Homo sapiens
FSNAWMSWVRQAPG

SEQ ID NO: 2 - protein, 19 aa, Homo sapiens
AFIWYDGSNKYYADSVKGR

SEQ ID NO: 3 - protein, 10 aa, Homo sapiens
ARYSGWYFDY

SEQ ID NO: 4 - protein, 15 aa, Homo sapiens
CTGSSSNIGAGYDVH

SEQ ID NO: 5 - protein, 7 aa, Homo sapiens
DNNNRPS

SEQ ID NO: 6 - protein, 12 aa, Homo sapiens
CQSYDSSLSAWL

SEQ ID NO: 7 - protein, 117 aa, Homo sapiens
EVQLLESGGGLVQPGGSLRLSCAASGFTFSNAWMSWVRQAPGKGLEWVAFIWYDGSNKYYADSVKGRFTISRDNSKNTLYLQMNSLRAEDTAVYYCARYSGWYFDYWGQGTLVTVSS

SEQ ID NO: 8 - protein, 112 aa, Homo sapiens
QSVLTQPPSASGTPGQRVTISCTGSSSNIGAGYDVHWYQQLPGTAPKLLIYDNNNRPSGVPDRFSGSKSGTSASLAISGLRSEDEADYYCQSYDSSLSAWLFGGGTKLTVLG

(19) United States
(12) Patent Application Publication
(10) Pub. No.: US 2014/0242072 A1
(43) Pub. Date: Aug. 28, 2014

(54) ANTI-ICAM-1 ANTIBODIES TO TREAT MULTIPLE-MYELOMA RELATED DISORDERS

(71) Applicant: BIOINVENT INTERNATIONAL AB, Lund (SE)

(72) Inventor: Markus Hansson, Eslov (SE)

(73) Assignee: BIOINVENT INTERNATIONAL AB, Lund (SE)

(21) Appl. No.: 14/347,636

(22) PCT Filed: Sep. 27, 2012
(86) PCT No.: PCT/EP2012/069132

§ 371 Date: Mar. 26, 2014

(30) Foreign Application Priority Data
Sep. 29, 2011 (GB) 1116774.9

(51) Int. Cl. C07K 16/28

(52) U.S. Cl. CPC C07K 16/2821
USPC 424/133.1; 530/389.1; 530/387.3; 424/172.1; 424/134.1; 530/388.2; 424/152.1; 530/388.15; 424/142.1; 424/135.1

(57) ABSTRACT

The invention relates to the use of an antibody or an antigen-binding fragment thereof with binding specificity for ICAM-1, or a variant, fusion or derivative of said antibody or an antigen-binding fragment with binding specificity for ICAM-1, for the treatment of a multiple-myeloma-related disorder. The invention also relates to methods for the administration of such antibodies, fragments, variants, fusion and derivatives thereof.

The invention relates to the use of an antibody or an antigen-binding fragment thereof with binding specificity for ICAM-1, or a variant, fusion or derivative of said antibody or an antigen-binding fragment with binding specificity for ICAM-1, for the treatment of a multiple-myeloma-related disorder. The invention also relates to methods for the administration of such antibodies, fragments, variants, fusion and derivatives thereof.

Multiple myeloma (also referred to as myeloma or MM) is a malignancy of B cells and accounts for 10% to 20% of total haematological malignancies. At present, it is an incurable disease with a median age at diagnosis of 65-70 years, and with very few patients diagnosed below the age of 40. In the United States, 19,920 new cases of multiple myeloma and more than 10,000 deaths are expected in 2008 to be myeloma-related (American Cancer Society, 2008). The disease has a slight male preponderance and is found more frequently in African Americans and less commonly in Asian populations (Kyle & Rajkumar, Blood. 2008 Mar. 15; 111(6):2962-72. Review).

A number of disorders are known to be related to multiple myeloma (in terms of their clinical presentation and their molecular and physiological basis) but are distinct disorders that can be distinguished from multiple myeloma. For example, like multiple myeloma many such related disorders arise from, or are characterised by, a clonal plasma cell disorder and patients afflicted with such disorders may exhibit one or more symptom known to occur in multiple myeloma. Such related disorders can therefore arise independently from multiple myeloma, or can present simultaneously with multiple myeloma (and either develop before or after the development of multiple myeloma). Accordingly, patients can have such multiple-myeloma-related disorders simultaneously with multiple myeloma or independently of multiple myeloma.

Three such multiple-myeloma-related disorders are Plasmacytoma (PC); Plasma Cell Leukemia (PCL); and Light Chain Amyloidosis (AL). There are currently no really effective treatments for multiple myeloma and consequently any current multiple myeloma treatments also do not provide an effective treatment for multiple-myeloma-related disorders such as PC, PCL and AL. In practice, current treatments for multiple-myeloma-related disorders rely on treating only the symptoms of the disorder (for example using pain killers) and do not treat the multiple-myeloma-related disorder itself.

The present invention provides a means for treating multiple-myeloma-related disorders.

In a first aspect, the invention provides:
an antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
for use in the treatment of a multiple-myeloma-related disorder, the treatment comprising the step of administering to a patient in need thereof an effective amount of the antibody, antigen-binding fragment, variant, fusion or derivative thereof to treat the multiple-myeloma-related disorder.
   In a second aspect, the invention provides:
use of an antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
in the manufacture of a medicament for the treatment of a multiple-myeloma-related disorder, the treatment comprising the step of administering to a patient in need thereof an effective amount of the antibody, antigen-binding fragment, variant, fusion or derivative thereof to treat the multiple-myeloma-related disorder.
   In a third aspect, the invention provides:
a method for treating a multiple-myeloma-related disorder in a patient, the method comprising the step of administering to a patient in need thereof an effective amount of:
an antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
wherein the amount of the antibody, antigen-binding fragment, variant, fusion or derivative thereof is effective to treat the multiple-myeloma-related disorder.

ICAM-1 is highly expressed on malignant and non-malignant cells and usually exists on the cell surface as a dimer. ICAM-1 is thought to be involved in cell-adhesion to bone-marrow stroma and, in malignant cells, thought to be associated with the development of drug resistance, angiogenesis and escape from immune surveillance.

WO 2007/068485 relates to an antibody capable of binding ICAM-1 on a cell surface and inducing cell death (for example, by induction of ADCC or apoptosis).

As discussed in the accompanying Examples, the present inventors have now surprisingly discovered that plasma cells present in patients having multiple-myeloma-related diseases express ICAM-1. That unexpected finding led to the development of the present invention involving the treatment of a multiple-myeloma-related disorder in a patient using an antibody or an antigen-binding fragment thereof with binding specificity for ICAM-1.

As will be appreciated by those skilled in the art, treatment with antibodies can offer therapeutic advantages with low toxicity in their ability to target cancerous cells and sparing surrounding tissues. The tolerability may reflect the dynamic actions of immunoglobulins, utilizing physiological mechanisms such as natural killer (NK)-cell mediated cell-death or directly inducing apoptosis rather than necrosis of tumour cells.

As non-cancerous (i.e. healthy) plasma cells also express ICAM-1, targeting that molecule will also reduce the number of non-cancerous plasma cells in an individual. Such cells are produced again from naïve B cells, which mature and differentiate to replace any healthy plasma cells that have been destroyed. Conversely, many other used to treat multiple myeloma (such as Rituximab) kill all CD20⁺ cells, which results in the killing of naïve B cells that are needed to regenerate healthy plasma cells in the individual.

Accordingly, the present invention provides a treatment with minimal side-effects and therefore offers a practical way to treat individuals with multiple-myeloma-related disorders.

By “a multiple-myeloma-related disorder” we include a disorder which is related to multiple myeloma in terms of its clinical presentation and molecular and physiological basis) but which is distinct from multiple myeloma and can be distinguished from it. For example, a multiple-myeloma-related disorder may have arisen from, or be characterised by, a clonal plasma cell disorder and patients afflicted with such a disorder may exhibit one or more symptom known to occur in multiple myeloma. A multiple-myeloma-related disorder may arise independently from multiple myeloma, or can be present simultaneously with multiple myeloma (and either develop before or after the development of multiple myeloma). Accordingly, patients can have such multiple-myeloma-related disorders simultaneously with multiple myeloma or independently of multiple myeloma.

As is well known to those skilled in the arts of medicine and oncology, multiple myeloma is a malignant, clonal disease originating from transformed plasma cells. A distinctive feature of the disease is that the malignant cells secrete monoclonal immunoglobulin (Ig), either in the form of IgG- or IgA-type (rarely IgD or IgE), or only light-chains (κ or λ), or both. A finding of monoclonal immunoglobulin (in blood or urine) is not mandatory, however, and in a small percentage of cases the myeloma is classified as “non-secretory”.

In recent years, substantial progress has been made in understanding the pathogenesis and molecular mechanisms of multiple myeloma. Genetic studies have revealed the occurrence of a vast array of different chromosomal changes, often carrying prognostic relevance, connected with this disease. Briefly, these chromosomal translocations often involve the immunoglobulin (Ig) H locus (14q32.3) and juxtaposes various transforming genes to segments promoted by the Ig enhancer, causing a dysregulated expression and potentially malignant transformation (Hideshima et al. Nat Rev Cancer. 2007. 7(8): 585-598).

Diagnostic criteria for MM include meeting three of the following criteria (Rajkumar (2011) American Journal of Hematology 86: 57-65):
Clonal bone marrow plasma cells at least 10%
Presence of serum and/or urinary monoclonal protein except in patients with true non-secretory multiple myeloma)
Evidence of end organ damage that can be attributed to the underlying plasma cell disorder (hypercalcemia, renal insufficiency, anemia, bone lesions)

In one embodiment, the patient having the multiple-myeloma-related disorder does not additionally have multiple-myeloma.

Preferably, the invention provides a use and method wherein the multiple-myeloma-related disorder is selected from the group comprising or consisting of: Plasmacytoma (PC); Plasma Cell Leukemia (PCL); Light Chain Amyloidosis (AL).

In one embodiment, the patient having one or more multiple-myeloma-related disorder selected from the group comprising or consisting of: Plasmacytoma (PC); Plasma Cell Leukemia (PCL); Light Chain Amyloidosis (AL), and the patient having that disorder does not additionally have multiple-myeloma.

Thus, in a preferred embodiment, the patient having Plasmacytoma does not additionally have multiple myeloma. In another preferred embodiment, the patient having Plasma Cell Leukemia does not additionally have multiple myeloma. In another preferred embodiment, the patient having Light Chain Amyloidosis does not additionally have multiple myeloma.

As is well known to those in the art, Plasmacytoma (for example, Solitary Plasmocytoma (SP)) is a symptomatic disorder characterised by monoclonal plasma cell proliferation in either bone or extramedullary soft tissue, but without evidence of significant bone-marrow plasma-cell infiltration. Bone Solitary Plasmocytoma is characterized by a unique lesion involving any part of the skeleton, most commonly the spine, and most often develops into multiple myeloma or additional solitary or multiple plasmocytomas (WHO, 2008). SP is generally treated by either radiation therapy (preferably) or surgical excision (rarely).

Diagnostic criteria for Plasmacytoma include meeting four of the following criteria (Rajkumar (2011) American Journal of Hematology 86: 57-65):
Biopsy proven solitary lesion of bone or soft tissue with evidence of clonal plasma cells
Normal bone marrow with no evidence of clonal plasma cells
Normal skeletal survey and MRI of spine and pelvis (except for the primary solitary lesion)
Absence of end-organ damage such as hypercalcemia, renal insufficiency, anemia, or bone lesions (CRAB) that can be attributed to a lymphoma-plasma cell proliferative disorder.

Plasmacytoma patients also typically exhibit no M-component in serum and/or urine. However, a small amount of M-component (less than 30 g/l) may sometimes occur (“Myelom utredning och behandling, nationella riktlinjer, diagnosgruppen for plasmacellssjukdomar” (“Myeloma Diagnosis and Treatment, National Guidelines and Diagnosis Group for Plasma Cell Disorders”), published in Swedish 28 Feb. 2010, revised 28 Feb. 2011, available from Svensk Förening för Hematologi (Swedish Society of Hematology, www.sfhem.se), for example, available at www.sfhem.se/content/download/3182/52091/file/Myelom%20riktlinjer%20version%202010 0228.pdf as of September 2011).

M-component is a protein comprising one or more antibody chain (such as a heavy or light chain, or both) and is produced and secreted by a plasma cell. Methods for detecting the presence of M-components in an individual are well known to those in the art and include serum and/or urine electrophoresis or immune fixation (or related immunochemical methods). Exemplary methods for detecting M-component are described in: National Comprehensive Cancer Network (NCCN) Clinical Practice Guidelines in Oncology: Multiple Myeloma. V.1.2008, National Comprehensive. Cancer Network, Inc. 2005/2006.

As is well known to those in the art, Plasma Cell Leukemia (PCL) is a rare, yet aggressive plasma cell neoplasm characterized by high levels of plasma cells circulating in the peripheral blood. PCL can either originate de novo (termed “primary PCL”) or as a secondary leukemic transformation of multiple myeloma (termed “secondary PCL”). Presenting signs and symptoms are similar to those seen in multiple myeloma such as renal insufficiency, hypercalcemia, lytic bone lesions, anemia, and thrombocytopenia, but can also include hepatomegaly (enlargement of the liver) and splenomegaly (enlargement of the spleen).

The diagnostic evaluation of a patient with suspected PCL should include a review of the peripheral blood smear, bone marrow aspiration and biopsy, serum protein electrophoresis (SPEP) with immunofixation, and protein electrophoresis of an aliquot from a 24 h urine collection (UPEP). The diagnosis can be made when a monoclonal population of PCs is present in the peripheral blood with an absolute PC count exceeding 2000 μL and PC comprising 20% or more of the peripheral blood white cells. The prognosis of PCL is poor with a median survival of 7 to 11 months.

In general, patients are treated with induction therapy followed by hematopoietic cell transplantation (HCT) in those who are appropriate candidates for this approach. The best induction regimen for PCL is not known and there is great variability in clinical practice. Newer agents that are being incorporated into frontline and salvage therapy for multiple myeloma have also demonstrated activity in PCL such as immunomodulatory agents and the use of bortezomib with different combinations.

As is well known to those in the art, Light chain amyloidosis (AL) is a clonal but non-proliferative plasma cell disorder in which fragments of an Ig light chain are deposited in tissues. The clinical features depend on the organs involved but can include restrictive cardiomyopathy, nephrotic syndrome, hepatic failure, and peripheral/autonomic neuropathy. AL is a severe and often fatal condition caused by pathological deposition of fibrillar aggregates of immunoglobulin light chains. Amyloidosis frequently manifests in organs such as kidneys, heart, skin, nervous system and in soft tissues, such as the tongue (Merlini and Belotti, 2003, NEJM, 349:583-596), and can result in organ failure. Organ failure typically involves heart and kidneys, resulting in severe cardiac arrhythmias or heart failure, and nephrotic syndrome, respectively.

Tissue biopsy stained with Congo red demonstrating amyloid deposits with apple-green birefringence is required for diagnosis. Invasive organ biopsy is not required because amyloid deposits can be found in bone marrow biopsy or subcutaneous fat aspirate in 85% of patients. N-terminal pro-brain natriuretic peptide and serum troponin T values are used to classify patients into three groups of approximately equal size; median survivals are 26.4, 10.5, and 3.5 months, respectively.

Diagnostic criteria for AL include meeting four of the following criteria (Rajkumar (2011) American Journal of Hematology 86: 57-65):
Presence of an amyloid-related systemic syndrome (such as renal, liver, heart, gastrointestinal tract or peripheral nerve involvement)
Positive amyloid staining by Congo red in any tissue (e.g. fat aspirate, bone marrow, or organ biopsy)
Evidence that amyloid is light-chain related established by direct examination of the amyloid (possibly using mass spectrometry-based proteomic analysis, or immuno-electron microscopy)
Evidence of a monoclonal plasma cell proliferative disorder (serum or M-protein, abnormal free light chain ratio, or clonal plasma cells in the bone marrow), (2-3% of patients with AL will not meet this requirement and must therefore be diagnosed with caution).

For amyloidosis, standard myeloma therapy has been used (as discussed in Comenzo, Blood. 2009. 114, 3147-3157) but the results are inferior to those in myeloma patients, which is believed to be due to the impaired organ function in amyloidosis. Amyloidosis of the heart, the most feared manifestation of this condition, has been regarded as an to irrevocably fatal disease (within ≦1 year) except for the rare cases where cardiac transplantation was an option.

As discussed above, there are currently no really effective treatments for multiple-myeloma-related disorders and current treatment relies on treating only the symptoms of the disorder (for example using pain killers) and does not treat the multiple-myeloma-related disorder itself. Accordingly, there exists a clear need for new treatments directed to individuals having multiple-myeloma-related disorders such as PC, PCL and AL. That need is addressed by the present invention which provides a treatment for the multiple-myeloma-related disorder itself without side effects associated with current treatments.

Details of the clinical and biochemical features of PC, PCL and AL and suitable tests and assays for identifying PC, PCL and AL, and for determining the characteristic criteria of those disorders, are known to those skilled in the art of medicine and are described above and in, for example, Rajkumar (2011) American Journal of Hematology 86: 57-65 and Albarracin and Fonseca (2011) Blood Reviews 25: 107-112).

In one embodiment, the invention provides an antibody, use or method in which the patient having the multiple-myeloma-related disorder additionally has multiple-myeloma.

In one embodiment, the patient having a multiple-myeloma-related disorder selected from the group comprising or consisting of: Plasmacytoma; Plasma Cell Leukemia; Light Chain Amyloidosis, and which patient additionally has multiple-myeloma.

By “additionally has multiple-myeloma” we include situations in which the patient having the multiple-myeloma-related disorder is additionally afflicted with (i.e. is suffering from) multiple myeloma. Such patients therefore represent a sub-group of multiple-myeloma patients as, in addition to multiple-myeloma, they are additionally afflicted with (i.e. suffer from) a multiple-myeloma-related disorder.

In one preferred embodiment, the patient has Plasmacytoma and multiple myeloma.

In a further preferred embodiment, the patient has Plasma Cell Leukemia and multiple myeloma.

In a further preferred embodiment, the patient has Light Chain Amyloidosis and multiple myeloma.

As discussed above, because the symptoms and clinical presentation of the multiple-myeloma-related disorders can be distinguished from multiple myeloma, a person skilled in the art will be capable of identifying patients afflicted with both a multiple-myeloma-related disorder (such as one or more of Plasmacytoma, Plasma Cell Leukemia and Light Chain Amyloidosis) and multiple myeloma itself.

It will be appreciated that the sub-group of patients suffering from both multiple myeloma and a multiple-myeloma-related disorder have a particularly serious and advanced medical condition and a poor prognosis.

For example, although the prognosis of Plasma Cell Leukemia alone is poor (with a median survival time of 7 to 11 months, as discussed above), patient survival is even shorter (between 2 to 7 months) when Plasma Cell Leukemia occurs in the context of multiple myeloma. Therefore, patients having both Plasma Cell Leukemia and multiple myeloma represent a sub-group of patients with a particularly aggressive plasma cell disorder, which is associated with a worse prognosis and shorter survival time than patients presenting with multiple myeloma alone (see, for example, Albarracin and Fonseca (2011) Blood Reviews 25: 107-112).

Similarly, Light Chain Amyloidosis is seen in 10-15% of patients with multiple myeloma and this sub-group of patients (i.e. patients having both Light Chain Amyloidosis and multiple myeloma) is considered consistent with an advanced form of multiple myeloma (“Myelom utredning och behandling, nationella riktlinjer, diagnosgruppen for plasmacellssjukdomar” 2010, revised 2011, as discussed above).

Plasmacytoma is a tumor mass consisting of atypical plasma cells. Incidence of plasmacytomas associated with multiple myeloma range from 7% to 17% at diagnosis and from 6% to 20% during the course of the disease. In both situations, occurrence of extramedullary disease has been consistently associated with poorer prognosis. Extramedullary relapse or progression occurs in a variety of clinical circumstances and settings, and therefore requires individualization of treatment (“Multiple myeloma with extramedullary disease”, Oriol A. (2011) Adv. Ther., Suppl. 7:1-6).

The present invention is particularly advantageous because it provides a treatment suitable for the patient sub-groups discussed above, which have particularly severe and aggressive forms of disease. As discussed in the accompanying Examples, the inventors' surprising finding that plasma cells present in patients having multiple-myeloma-related diseases (such as Plasmacytoma, Plasma Cell Leukemia and Light Chain Amyloidosis) express ICAM-1 provides a means for treating such disorders.

Previous approaches have treated symptoms arising in such patient sub-groups but not treated the underlying disorder itself, resulting in a very poor prognosis and short survival time. Indeed, in some cases, such patient sub-groups may have been regarded as having such severe disease that they have been deemed not worth treating using conventional therapies.

By “treatment” we include both therapeutic and prophylactic treatment of a subject/patient. The term “prophylactic” is used to encompass the use of an antibody, medicament or composition described herein which either prevents or reduces the likelihood of the occurrence or development of a condition or disorder (such as a multiple-myeloma-related disorder) in an individual.

By “antibody” we include substantially intact antibody molecules, as well as chimeric antibodies, humanised antibodies, human antibodies (wherein at least one amino acid is mutated relative to the naturally occurring human antibodies), single chain antibodies, bi-specific antibodies, antibody heavy chains, antibody light chains, homo-dimers and heterodimers of antibody heavy and/or light chains, and antigen binding fragments and derivatives of the same.

The term “antibody” also includes all classes of antibodies, including IgG, IgA, IgM, IgD and IgE. Thus, the antibody may be an IgG molecule, such as an IgG1, IgG2, IgG3, or IgG4 molecule. Preferably, the antibody of the invention is an IgG molecule, or an antigen-binding fragment, or variant, fusion or derivative thereof.

The methods and uses of the invention encompass variants, fusions and derivatives of the defined antibodies and antigen-binding fragments thereof, as well as fusions of a said variants or derivatives, provided such variants, fusions and derivatives have binding specificity for ICAM-1.

As antibodies and antigen-binding fragments thereof comprise one or more polypeptide component, variants, fusions and derivatives of the antibody and antigen-binding fragment thereof as defined herein may be made using the methods of protein engineering and site-directed mutagenesis well known in the art, using the recombinant polynucleotides (see example, see Molecular Cloning: a Laboratory Manual, 3rd edition, Sambrook & Russell, 2001, Cold Spring Harbor Laboratory Press, which is incorporated herein by reference).

Thus, variants, fusions and derivatives of the antibody or antigen-binding fragment thereof as defined herein, may be made based on the polypeptide component of the antibody or antigen-binding fragment thereof.

By “fusion” we include said polypeptide fused to any other polypeptide. For example, the said polypeptide may be fused to a polypeptide such as glutathione-S-transferase (GST) or protein A in order to facilitate purification of said polypeptide. Examples of such fusions are well known to those skilled in the art. Similarly, the said polypeptide may be fused to an oligo-histidine tag such as His6 or to an epitope recognised by an antibody such as the well-known Myc-tag epitope. Fusions to any variant or derivative of said polypeptide are also included in the scope of the invention. It will be appreciated that fusions (or variants or derivatives thereof) which retain desirable properties, such as have binding specificity for ICAM-1, are preferred.

The fusion may comprise or consist of a further portion which confers a desirable feature on the said polypeptide; for example, the portion may be useful in detecting or isolating the polypeptide, or promoting cellular uptake of the polypeptide. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc-tag, as known to those skilled in the art or may be a lipophilic molecule or polypeptide domain that is capable of promoting cellular uptake of the polypeptide, as known to those skilled in the art.

By “variants” of said polypeptide we include insertions, deletions and substitutions, either conservative or non-conservative. In particular we include variants of the polypeptide where such changes do not substantially alter the activity of the said polypeptide. In particular, we include variants of the polypeptide where such changes do not substantially alter the binding specificity for ICAM-1.

The polypeptide variant may have an amino acid sequence which has at least 75% identity with one or more of the amino acid sequences given above, for example at least 80%, at least 90%, at least 95%, at least 96%, at least 97%, at least 98%, or at least 99% identity with one or more of the amino acid sequences specified above.

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally.

The alignment may alternatively be carried out using the Clustal W program (as described in Thompson et al., 1994, Nucl. Acid Res. 22:4673-4680, which is incorporated herein by reference).

The parameters used may be as follows:
Fast pair-wise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

The antibody, antigen-binding fragment, variant, fusion or derivative used in the methods or uses of the invention may comprise or consist of one or more amino acids which have been modified or derivatised.

Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides, which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylanidation (e.g. with ammonia or methylamine), and the like terminal modifications.

It will be further appreciated by persons skilled in the art that peptidomimetic compounds may also be useful. Thus, by ‘polypeptide’ we include peptidomimetic compounds which are capable of binding ICAM-1. The term ‘peptidomimetic’ refers to a compound that mimics the conformation and desirable features of a particular peptide as a therapeutic agent.

For example, the said polypeptide includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al., (1997) J. Immunol. 159, 3230-3237, which is incorporated herein by reference. This approach involves making pseudo-peptides containing changes involving the backbone, and not the orientation of side chains. Retro-inverse peptides, which contain NH—CO bonds instead of CO—NH peptide bonds, are much more resistant to proteolysis. Alternatively, the said polypeptide may be a peptidomimetic compound wherein one or more of the amino acid residues are linked by a -y(CH₂NH)— bond in place of the conventional amide linkage.

In a further alternative, the peptide bond may be dispensed with altogether provided that an appropriate linker moiety which retains the spacing between the carbon atoms of the amino acid residues is used; it may be advantageous for the linker moiety to have substantially the same charge distribution and substantially the same planarity as a peptide bond.

It will be appreciated that the said polypeptide may conveniently be blocked at its N- or C-terminus so as to help reduce susceptibility to exo-proteolytic digestion.

A variety of un-coded or modified amino acids such as D-amino acids and N-methyl amino acids have also been used to modify mammalian peptides. In addition, a presumed bioactive conformation may be stabilised by a covalent modification, such as cyclisation or by incorporation of lactam or other types of bridges, for example see Veber et al., 1978, Proc. Natl. Acad. Sci. USA 75:2636 and Thursell at al., 1983, Biochem. Biophys. Res. Comm. 111:166, which are incorporated herein by reference.

A common theme among many of the synthetic strategies has been the introduction of some cyclic moiety into a peptide-based framework. The cyclic moiety restricts the conformational space of the peptide structure and this frequently results in an increased specificity of the peptide for a particular biological receptor. An added advantage of this strategy is that the introduction of a cyclic moiety into a peptide may also result in the peptide having a diminished sensitivity to cellular peptidases.

Thus, exemplary polypeptides useful in the methods and uses of the invention comprise or consist of terminal cysteine amino acids. Such a polypeptide may exist in a heterodetic cyclic form by disulphide bond formation of the mercaptide groups in the terminal cysteine amino acids or in a homodetic form by amide peptide bond formation between the terminal amino acids. As indicated above, cyclising small peptides through disulphide or amide bonds between the N- and C-terminus cysteines may circumvent problems of specificity and half-life sometime observed with linear peptides, by decreasing proteolysis and also increasing the rigidity of the structure, which may yield higher specificity compounds. Polypeptides cyclised by disulphide bonds have free amino and carboxy-termini which still may be susceptible to proteolytic degradation, while peptides cyclised by formation of an amide bond between the N-terminal amine and C-terminal carboxyl and hence no longer contain free amino or carboxy termini. Thus, the peptides can be linked either by a C—N linkage or a disulphide linkage.

The present invention is not limited in any way by the method of cyclisation of peptides, but encompasses peptides whose cyclic structure may be achieved by any suitable method of synthesis. Thus, heterodetic linkages may include, but are not limited to formation via disulphide, alkylene or sulphide bridges. Methods of synthesis of cyclic homodetic peptides and cyclic heterodetic peptides, including disulphide, sulphide and alkylene bridges, are disclosed in U.S. Pat. No. 5,643,872, which is incorporated herein by reference. Other examples of cyclisation methods are discussed and disclosed in U.S. Pat. No. 6,008,058, which is incorporated herein by reference.

A further approach to the synthesis of cyclic stabilised peptidomimetic compounds is ring-closing metathesis (RCM). This method involves steps of synthesising a peptide precursor and contacting it with an RCM catalyst to yield a conformationally restricted peptide. Suitable peptide precursors may contain two or more unsaturated C—C bonds. The method may be carried out using solid-phase-peptide-synthesis techniques. In this embodiment, the precursor, which is anchored to a solid support, is contacted with a RCM catalyst and the product is then cleaved from the solid support to yield a conformationally restricted peptide.

Another approach, disclosed by D. H. Rich in Protease Inhibitors, Barrett and Selveson, eds., Elsevier (1986), which is incorporated herein by reference, has been to design peptide mimics through the application of the transition state analogue concept in enzyme inhibitor design. For example, it is known that the secondary alcohol of staline mimics the tetrahedral transition state of the scissile amide bond of the pepsin substrate.

In summary, terminal modifications are useful, as is well known, to reduce susceptibility by proteinase digestion and therefore to prolong the half-life of the peptides in solutions, particularly in biological fluids where proteases may be present. Polypeptide cyclisation is also a useful modification because of the stable structures formed by cyclisation and in view of the biological activities observed for cyclic peptides.

Thus, in one embodiment the said polypeptide is cyclic. However, in an alternative embodiment, the said polypeptide is linear.

By “binding specificity for ICAM-1” we mean an antibody or antigen-binding fragment, or variant, fusion or derivative thereof, which is capable of binding to ICAM-1 selectively. By “capable of binding selectively” we include such antibody-derived binding moieties which bind at least 10-fold more strongly to ICAM-1 than to another proteins; for example at least 50-fold more strongly, or at least 100-fold more strongly. The binding moiety may be capable of binding selectively to ICAM-1 under physiological conditions, e.g. in vivo.

Such binding specificity may be determined by methods well known in the art, such as enzyme-linked immunosorbent assay (ELISA), immunohistochemistry, immunoprecipitation, Western blot and flow cytometry using transfected cells expressing ICAM-1. Suitable methods for measuring relative binding strengths include immunoassays, for example where the binding moiety is an antibody (see Harlow & Lane, “Antibodies: A Laboratory Manual”, Cold Spring Habor Laboratory Press, New York, which is incorporated herein by reference). Alternatively, binding may be assessed using competitive assays or using Biacore® analysis (Biacore International AB, Sweden).

In a further embodiment, the antibody or antigen-binding fragment, or variant, fusion or derivative thereof, binds exclusively to ICAM-1.

It will be appreciated by persons skilled in the art that the binding specificity of an antibody or antigen binding fragment thereof is conferred by the presence of complementarity determining regions (CDRs) within the variable regions of the constituent heavy and light chains. As discussed below, in a particularly preferred embodiment of the antibodies and antigen-binding fragments, variants, fusions and derivatives thereof defined herein, binding specificity for ICAM-1 is conferred by the presence of one or more of the six CDRs identified as SEQ ID NOS: 1 to 6 herein.

In a preferred embodiment, the antibody or antigen-binding fragment, or variant, fusion or derivative thereof of the antibody defined herein retains the binding specificity for ICAM-1 of the original antibody. By “retains the binding specificity” we mean that the antibody or antigen-binding fragment, or variant, fusion or derivative thereof as defined herein, is capable of competing for binding to ICAM-1 with the exemplary antibody of the invention (designated BI-AB; see accompanying Examples). For example, the antibody or antigen-binding fragment, or variant, fusion or derivative thereof, may bind to the same epitope on ICAM-1 as an antibody comprising or consisting of the CDRs identified as SEQ ID NOS: 1 to 6.

By “epitope” it is herein intended to mean a site of a molecule to which an antibody binds, i.e. a molecular region of an antigen. An epitope may be a linear epitope, which is determined by e.g. the amino acid sequence, i.e. the primary structure, or a three-dimensional epitope, defined by the secondary structure, e.g. folding of a peptide chain into beta sheet or alpha helical, or by the tertiary structure, e.g. way which helices or sheets are folded or arranged to give a three-dimensional structure, of an antigen.

Methods for determining whether a test antibody is capable of competing for binding with second antibody are well known in the art (such as, for example sandwich-ELISA or reverse-sandwich-ELISA techniques) and described, for example, in Antibodies: A Laboratory. Manual, Harlow & Lane (1988, CSHL, NY, ISBN 0-87969-314-2), which is incorporated herein by reference.

The antibody or antigen-binding fragment, or variant, fusion or derivative thereof, with binding specificity for ICAM-1 may also retain one or more of the same biological properties as the original antibody (such as the exemplary antibody, BI-AB).

Intercellular Adhesion Molecule-1 (“ICAM-1”; also referred to as CD54) is an 80-114 kDa glycosylated cell-surface transmembrane protein consisting of five immunoglobulin domains, a transmembrane domain and a short cytoplasmic domain.

ICAM-1 functions as a ligand for leukocyte function associated antigen-1 (CD11a/C418), and additionally binds Mac-1 (CD11b/CD18), CD43, MUC-1, rhinovirus and fibrinogen. The predominant function of ICAM-1 is the recruitment of leucocytes to inflammatory sites, but ICAM-1 also participates in other cell-cell adhesions and in cell-activation, cell-signalling, cell-migration and cell-invasion (Rosette et al. Carcinogenesis 26, 943-950 (2005); Gho et al., Cancer Res 59, 5128-5132 (1999); Gho et al., Cancer Res 61, 4253-4257 (2001); Chirathaworn et al. J Immunol 168, 5530-5537 (2002); Berg et al., J Immunol 155, 1694-1702 (1995); Martz, Hum Immunol 18, 3-37 (1987); Poudrier & Owens, J. Exp. Med. 179, 1417-1427 (1994); Sun et al. J Cancer Res Clin Oncol 125, 28-34 (1999); Springer, Cell 76, 301-314 (1994); Siu et al., J Immunol 143, 3813-3820 (1989); Dang et al., J Immunol 144, 4082-4091 (1990); Damle et al. J Immunol 151, 2368-2379 (1993); Lane et al., J Immunol 147, 4103-4108 (1991); Ybarrondo et al., J Exp Med 179, 359-363 (1994)).

The importance of ICAM-1 in these processes is complex and has been the subject of numerous investigations. Most frequently ICAM-1 deficiency, whether conferred by genetic ablation, siRNA administration or function-blocking antibodies, has been shown to interfere with leukocyte extravasation and recruitment to inflammatory sites by decreasing or delaying cell migration and activation to different extents (Reilly et al. J Immunol 155, 529-532 (1995); Kim et al. J Immunother (1997) 30, 727-739 (2007); Smallshaw et al., J Immunother 27, 419-424 (2004); Coleman et al., J Immunother 29, 489-498 (2006); Kawano et al. Br J Haematol 79, 583-588 (1991)).

ICAM-1 exists on the cell surface as a dimer, but can also multimerize in the form of W-shapes, rings or long chains (Springer, Cell 76, 301-314 (1994); Siu, et al., J Immunol 143, 3813-3820 (1989). Thus, by “ICAM-1” we include the monomeric form of the molecule, and dimers and multimers of the ICAM-1 monomer, including multimers in the form of W-shapes, rings or long chains.

It will be appreciated by persons skilled in the art that ICAM-1 may be derived from a human or non-human animal. In one embodiment, ICAM-1 is human.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host.

The agents (i.e. antibody, antigen-binding fragment, variant, fusion or derivative thereof), medicaments and pharmaceutical compositions of the invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period. Preferably, delivery is performed intra-muscularly (i.m.) and/or sub-cutaneously (s.c.) and/or intravenously (i.v.).

The agents, medicaments and pharmaceutical compositions of the invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Preferably, the medicaments and/or pharmaceutical compositions of the present invention is a unit dosage containing a daily dose or unit, daily sub-dose or an appropriate fraction thereof, of the active ingredient.

The agents, medicaments and pharmaceutical compositions of the invention will normally be administered by a parenteral route, in the form of a pharmaceutical composition comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the agents, medicaments and pharmaceutical compositions of the invention can be administered alone but will generally be administered in admixture with a suitable pharmaceutical excipient, diluent or carrier selected with regard to the intended route of administration and standard pharmaceutical practice.

The agents, medicaments and pharmaceutical compositions of the invention can be administered parenterally, for example, intravenously, intra-arterially, intraperitoneally, intra-thecally, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Medicaments and pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The medicaments and pharmaceutical compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For parenteral administration to human patients, the daily dosage level of the agents, medicaments and pharmaceutical compositions of the invention will usually be from 1 μg to 10 mg per adult per day administered in single or divided doses. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

Typically, the medicaments and pharmaceutical compositions of the invention will contain the agent of the invention at a concentration of between approximately 2 mg/ml and 150 mg/ml or between approximately 2 mg/ml and 200 mg/ml. In a preferred embodiment, the medicaments and pharmaceutical compositions of the invention will contain the agent of the invention at a concentration of 10 mg/ml.

Generally, in humans, oral or parenteral administration of the agents, medicaments and pharmaceutical compositions of the invention is the preferred route, being the most convenient.

For veterinary use, the agents, medicaments and pharmaceutical compositions of the invention are administered as a suitably acceptable formulation in accordance with normal veterinary practice and the veterinary surgeon will determine the dosing regimen and route of administration which will be most appropriate for a particular animal.

Thus, the present invention provides a pharmaceutical formulation comprising an amount of an antibody or antigen-binding fragment, or variant, fusion or derivative thereof, of the invention effective to treat various conditions (as described above and further below).

Preferably, the pharmaceutical composition is adapted for delivery by a route selected from the group comprising: intravenous; intramuscular; subcutaneous.

The present invention also includes pharmaceutical compositions comprising pharmaceutically acceptable acid or base addition salts of the polypeptide binding moieties of the present invention. The acids which are used to prepare the pharmaceutically acceptable acid addition salts of the aforementioned base compounds useful in this invention are those which form non-toxic acid addition salts, i.e. salts containing pharmacologically acceptable anions, such as the hydrochloride, hydrobromide, hydroiodide, nitrate, sulphate, bisulphate, phosphate, acid phosphate, acetate, lactate, citrate, acid citrate, tartrate, bitartrate, succinate, maleate, fumarate, gluconate, saccharate, benzoate, methanesulphonate, ethanesulphonate, benzenesulphonate, p-toluenesulphonate and pamoate [i.e. 1,1′-methylene-bis-(2-hydroxy-3 naphthoate)] salts, among others.

Pharmaceutically acceptable base addition salts may also be used to produce pharmaceutically acceptable salt forms of the agents according to the present invention.

The chemical bases that may be used as reagents to prepare pharmaceutically acceptable base salts of the present agents that are acidic in nature are those that form non-toxic base salts with such compounds. Such non-toxic base salts include, but are not limited to those derived from such pharmacologically acceptable cations such as alkali metal cations (e.g. potassium and sodium) and alkaline earth metal cations (e.g. calcium and magnesium), ammonium or water-soluble amine addition salts such as N-methylglucamine-(meglumine), and the lower alkanolammonium and other base salts of pharmaceutically acceptable organic amines, among others.

The agents and/or polypeptide binding moieties of the invention may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of antibody activity loss (e.g. with conventional immunoglobulins, IgM antibodies tend to have greater activity loss than IgG antibodies) and that use levels may have to be adjusted upward to compensate. In one embodiment, the lyophilised (freeze dried) polypeptide binding moiety loses no more than about 20%, or no more than about 25%, or no more than about 30%, or no more than about 35%, or no more than about 40%, or no more than about 45%, or no more than about 50% of its activity (prior to lyophilisation) when re-hydrated.

Preferably, the invention provides an antibody, use or method wherein the effective amount of the antibody, antigen-binding fragment, variant, fusion or derivative thereof is between about 0.0001 mg/kg to 50 mg/kg of the antibody, antigen-binding fragment, variant, fusion or derivative thereof.

As is appreciated by those skilled in the art, the precise amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition; complications, other diseases, etc., as is well known in the art.

In one embodiment, the ICAM-1 to which the antibody or antigen-binding fragment, or a variant, fusion or derivative thereof, of the invention, binds is localised on the surface of a plasma cell.

By “plasma cell”, we include a cell derived from the clonal expansion of a differentiated B cell and which is capable of synthesising an antibody molecule.

ICAM-1 is expressed constitutively at low levels on vascular endothelium, epithelial cells, fibroblasts, keratinocytes, leukocytes as well as conventional antigen presenting cells (APC) (reviewed in Roebuck. & Finnegan, J Leukoc Biol, 66, 876-888 (1999)). Cell surface ICAM-1 expression can be induced by several cytokines and pro-inflammatory agents, such as IFN-γ, TNF-α, lipopolysaccharide (LPS), oxygen radicals or hypoxia (Roebuck & Finnegan, J Leukoc Biol 66, 876-888 (1999)). ICAM-1 may additionally be shed from cells by proteolysis or by alternative splicing depending on cell type (Dang et al., J Immunol 144, 4082-4091 (1990); Damle et al. J Immunol 151, 2368-2379 (1993); Lane et al., J Immunol 147, 4103-4108 (1991); Ybarrondo, et al., J Exp Med 179, 359-363 (1994)).

It is preferred that the antibody or antigen-binding fragment, or variant, fusion or derivative thereof, is capable of specifically binding ICAM-1 localised on the surface of a cell and inhibiting and/or preventing proliferation of that cell.

By “proliferation”, we include the growth of an individual cell and its division into daughter cells. Suitable assays for testing cellular proliferation (both in vitro and in vivo) are known in the art.

In one embodiment, the antibody or antigen-binding fragment, or variant, fusion or derivative thereof, is capable of specifically binding ICAM-1 localised on the surface of a cell and inducing apoptosis of that cell. Apoptosis is thought to be initiated by antibody binding to, and cross-linking, ICAM-1 localised on the cell surface.

As is well known, apoptosis is a form of cell-death in which a programmed sequence of events leads to the elimination of cells without releasing harmful substances into the surrounding environment. During the normal functioning of an individual, apoptosis plays a crucial role in developing and maintaining health by eliminating old, unnecessary and/or unhealthy cells. Thus, by “inducing apoptosis” of a cell we mean that the cell is eliminated by the induction of apoptosis. Suitable assays for measuring apoptosis (both in vitro or in vivo) are known in the art.

In the present invention, apoptosis of plasma cells associated with, or responsible for, the multiple-myeloma-related disorder will prevent continuance of that disorder.

In a further embodiment, the antibody or antigen-binding fragment, or variant, fusion or derivative thereof, is capable of specifically binding ICAM-1 localised on the surface of a cell and inducing antibody-dependent cell cytotoxicity (ADCC) against that cell.

As is well known, ADCC is an immune response in which antibodies bind to a target cell, leading to elimination of that targeted cell by the immune system. Suitable assays for measuring ADCC (both in vitro and in vivo) are known in the art. In the present invention, ADCC-mediated elimination of plasma cells associated with, or responsible for, the multiple-myeloma-related disorder will prevent continuance of that disorder.

Preferably, the antibody or antigen-binding fragment has efficacy in the treatment of a multiple-myeloma-related disorder.

Preferably, the antibody or antigen-binding fragment, or a variant, fusion or derivative thereof, comprises or consists of an intact antibody. Alternatively, the antibody or antigen-binding fragment, or variant, fusion or derivative thereof, may consist essentially of an intact antibody. By “consist essentially of” we mean that the antibody or antigen-binding fragment, variant, fusion or derivative thereof consists of a portion of an intact antibody sufficient to display binding specificity for ICAM-1.

In one embodiment of the uses and methods of the invention, the antibody is a non-naturally occurring antibody. Of course, where the antibody is a naturally occurring antibody, it is provided in an isolated form (i.e. distinct from that in which it is found in nature).

In an alternative embodiment, the antibody or antigen-binding fragment, or a variant, fusion or derivative thereof, comprises or consists of an antigen-binding fragment selected from the group consisting of: an Fv fragment; an Fab fragment; an Fab-like fragment.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent-parented antibody (Morrison et al (1984) Proc. Natl. Acad. Sci. USA 81, 6851-6855).

Antigenic specificity is conferred by variable domains and is independent of the constant domains, as known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising or consisting of isolated V domains (Ward et al (1989) Nature 341, 544). A general review of the techniques involved in the synthesis of antibody fragments which retain their specific binding sites is to be found in Winter & Milstein (1991) Nature 349, 293-299.

Thus, by “antigen-binding fragment” we include a functional fragment of an antibody that is capable of binding to ICAM-1.

Exemplary antigen-binding fragments may be selected from the group consisting of Fv fragments (e.g. single chain Fv and disulphide-bonded Fv), and Fab-like fragments (e.g. Fab fragments, Fab′ fragments and F(ab)₂ fragments).

In one embodiment of the uses and methods of the invention, the antigen-binding fragment is an scFv.

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

Also included within the scope of the invention are modified versions of antibodies and an antigen-binding fragments thereof, e.g. modified by the covalent attachment of polyethylene glycol or other suitable polymer.

It is particularly preferred that the antibody or antigen-binding fragment, or the variant, fusion or derivative thereof is recombinant molecule.

Methods of generating antibodies and antibody fragments are well known in the art. For example, antibodies may be generated via any one of several methods which employ induction of in vivo production of antibody molecules, screening of immunoglobulin libraries (Orlandi et al, 1989. Proc. Natl. Acad. Sci. U.S.A. 86:3833-3837; Winter et al., 1991, Nature 349:293-299) or generation of monoclonal antibody molecules by cell lines in culture. These include, but are not limited to, the hybridoma technique, the human B-cell hybridoma technique, and the Epstein-Barr virus (EBV)-hybridoma technique (Kohler at al., 1975. Nature 256:4950497; Kozbor et al., 1985. J. Immund. Methods 81:31-42; Cote et al., 1983. Proc. Natl. Acad. Sci. USA 80:2026-2030; Cole et al., 1984. Mol. Cell. Biol. 62:109-120).

Conveniently, the invention provides an antibody or antigen-binding fragment, or a variant, fusion or derivative thereof, wherein the antibody is a recombinant antibody (i.e. wherein it is produced by recombinant means).

In a particularly preferred embodiment, the invention provides a antibody, use or method in which the antibody is a monoclonal antibody.

Suitable monoclonal antibodies to selected antigens may be prepared by known techniques, for example those disclosed in “Monoclonal Antibodies: A manual of techniques” H Zola (CRC Press, 1988) and in “Monoclonal Hybridoma Antibodies: Techniques and Applications”, J G R Hurrell (CRC Press, 1982), which are incorporated herein by reference.

Antibody fragments can also be obtained using methods well known in the art (see, for example, Harlow & Lane, 1988, “Antibodies: A Laboratory Manual”, Cold Spring Harbor Laboratory, New York, which is incorporated herein by reference). For example, antibody fragments for use in the methods and uses of the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

Preferably, the antibody or antigen-binding fragment thereof is a human antibody or humanised antibody.

Preferably, the invention provides an antibody, use or method wherein the antibody or antigen-binding fragment thereof is a human antibody or humanised antibody.

It will be appreciated by persons skilled in the art that for human therapy or diagnostics, humanised antibodies may be used. Humanised forms of non-human (e.g. murine) antibodies are genetically engineered chimeric antibodies or antibody fragments having minimal-portions derived from non-human antibodies. Humanised antibodies include antibodies in which complementary determining regions of a human antibody (recipient antibody) are replaced by residues from a complementary determining region of a non human species (donor antibody) such as mouse, rat of rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann at al., 1988, Nature 332:323-329; Presta, 1992, Curr. Op. Struct. Biol. 2:593-596, which are incorporated herein by reference).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-1536I; U.S. Pat. No. 4,816,567, which are incorporated herein by reference), by substituting human complementarity determining regions with corresponding rodent complementarity determining regions. Accordingly, such humanised antibodies are chimeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, J. Mol. Biol. 227:381; Marks et al., 1991, J. Mol. Biol. 222:581; Cole et al., 1985, In: Monoclonal antibodies and Cancer Therapy, Alan R. Liss, pp. 77; Boerner et al., 1991. J. Immunol. 147:86-95, Soderlind et al., 2000, Nat Biotechnol 18:852-6 and WO 98/32845 which are incorporated herein by reference).

In one embodiment, the antibody or antigen-binding fragment thereof comprises or consists of one or more of the following amino acid sequences:

[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

It will be appreciated that the amino acid sequences of SEQ ID NO: 1 to 6 represent antibody Complementarity Determining Regions (CDRs).

The term “amino acid” as used herein includes the standard twenty genetically-encoded amino acids and their corresponding stereoisomers in the ‘D’ form (as compared to the natural ‘L’ form), omega-amino acids other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as “alanine” or “Ala” or “A”, the term refers to both L-alanine and D-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid.

In one embodiment, the polypeptides as defined herein comprise or consist of L-amino acids.

Preferably, the heavy chain variable region of the antibody, fragment, variant, fusion or derivative comprises or consists of the following CDRs:

[SEQ ID NO: 1]
FSNAWMSWVRQAPG;
and
[SEQ ID NO: 2]
AFIWYDGSNKYYADSVKGR;
and
[SEQ ID NO: 3]
ARYSGWYFDY.

In one embodiment, the heavy chain variable region of the antibody, fragment, variant, fusion or derivative comprises or consists of the amino acid sequence of SEQ ID NO: 7.

[SEQ ID NO: 7]
EVQLLESGGGLVQPGGSLRLSCAASGFTFSNAWMSWVRQAPGKGLEWVAF
IWYDGSNKYYADSVKGRFTISRDNSKNTLYLQMNSLRAEDTAVYYCARYS
GWYFDYWGQGTLVTVSS

Preferably, the light chain variable region of the antibody, fragment, variant, fusion or derivative comprises or consists of the following CDRs:

[SEQ ID NO: 4]
CTGSSSNIGAGYDVH;
and
[SEQ ID NO: 5]
DNNNRPS;
and
[SEQ ID NO: 6]
CQSYDSSLSAWL.

In one embodiment, the light chain variable region of the antibody, fragment, variant, fusion or derivative comprises or consists of the amino acid sequence of SEQ ID NO: 8.

[SEQ ID NO: 8]
QSVLTQPPSASGTPGQRVTISCTGSSSNIGAGYDVHWYQQLPGTAPKLLI
YDNNNRPSGVPDRFSGSKSGTSASLAISGLRSEDEADYYCQSYDSSLSAW
LFGGGTKLTVLG

In a particularly preferred embodiment, the antibody or antigen-binding fragment, variant, fusion or derivative comprises or consists of a heavy chain variable region as defined herein and a light chain variable region as defined herein.

For example, the antibody or antigen-binding fragment, variant, fusion or derivative may comprise or consist of a heavy chain variable region comprising or consisting of the amino acid sequence of SEQ ID NO:7 and a light chain variable region comprising or consisting of the amino acid sequence of SEQ ID NO:8.

As discussed above, in a preferred embodiment, the antibody or antigen-binding fragment, or variant, fusion or derivative thereof is capable of competing for binding to ICAM-1 with the exemplary antibody of the invention (designated BI-AB; see accompanying Examples). For example, the antibody or antigen-binding fragment, or variant, fusion or derivative thereof, may bind to the same epitope on ICAM-1 as an antibody comprising or consisting of the CDRs identified as SEQ ID NOS: 1 to 6. Methods for determining whether a test antibody is capable of competing for binding with second antibody are well known in the art and have been discussed above.

In one embodiment, invention provides an antibody, use or method as defined herein wherein the antibody or antigen-binding fragment, variant, fusion or derivative thereof is capable of competing for binding to ICAM-1 with: an antibody or antigen-binding fragment, variant, fusion or derivative thereof which comprises or consists of a heavy chain variable region comprising or consisting of the CDRs of SEQ ID NO:1-3 and a light chain variable region, comprising or consisting of the CDRs of SEQ ID NO:4-6; or an antibody or antigen-binding fragment, variant, fusion or derivative thereof which comprises or consists of a heavy chain variable region comprising or consisting of the amino acid sequence of SEQ ID NO:7 and a light chain variable region comprising or consisting of the amino acid sequence of SEQ ID NO:8.

In one embodiment, said antibody or antigen binding fragment is derived from an n-CoDeR® antibody library. That library is further described in WO 98/32845 (to BioInvent International AB) and is incorporated herein by reference.

Once suitable antibodies are obtained, they may be tested for activity, such as binding specificity or a biological activity of the antibody, for example by ELISA, immunohistochemistry, flow cytometry, immunoprecipitation, Western blots, etc. The biological activity may be tested in different assays with readouts for that particular feature.

In a further aspect, the invention provides a kit comprising an agent or a pharmaceutical composition as defined herein. Thus, there may be provided a kit for use in the therapeutic treatment of the conditions defined herein.

Alternatively, the kit may comprise a detectable antibody or antigen-binding fragment or derivative thereof according to the invention, suitable for use in diagnosis. Such a diagnostic kit may comprise, in an amount sufficient for at least one assay, the diagnostic agent as a separately packaged reagent. Instructions for use of the packaged reagent are also typically included. Such instructions typically include a tangible expression describing reagent concentrations and/or at least one assay method parameter such as the relative amounts of reagent and sample to be mixed, maintenance time periods for reagent/sample admixtures, temperature, buffer conditions and the like.

As used herein, the singular forms “a”, “and”, and “the” include plural referents unless the context clearly dictates otherwise. Thus, for example, reference to “an antibody” includes a plurality of such antibodies and reference to “the dosage” includes reference to one or more dosages and equivalents thereof known to those skilled in the art, and so forth.

Preferred, non-limiting examples which embody certain aspects of the invention will now be described, with reference to the following figures:

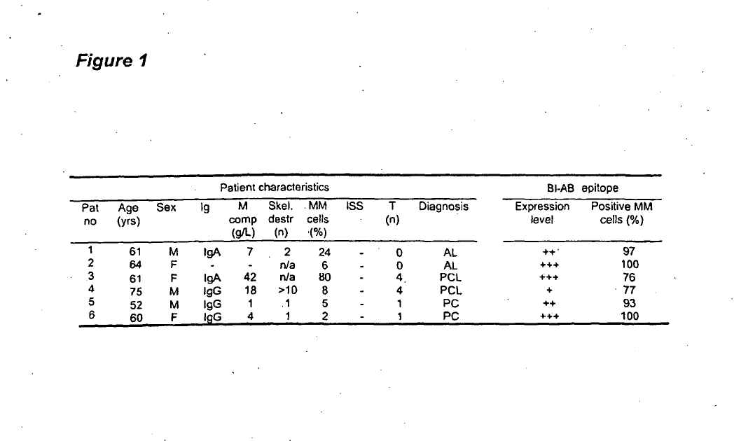

FIG. 1: BI-AB epitope expression in a cohort of patients with plasma cell disorders, consecutively analysed at Lund University Hospital, Sweden.
Pat no (Patient number); M (male); F (female); Ig (immunoglobulin class of M-component); M comp (monoclonal Ig component in serum); Skel. destr (number of skeletal destruction as measured by X-ray); n/a (not available); MM-cells (cells from multiple-myeloma-related disorders counted as % of all nucleated cells in bone marrow smears); ISS (International Staging System of MM); T (number of different MM treatment regimes before BI-AB analysis); Diagnosis; AL (amyloid light chain amyloidosis); PCL (plasma cell leukemia); PC (plasmacytoma); Expression level (BI-AB epitope expression level measured by FACS on cells from multiple-myeloma-related disorders: +++ (very highly positive), ++ (highly positive), + (positive) or − (negative); Positive MM cells (BI-AB epitope positive cells from multiple-myeloma-related disorders as measured by FACS).

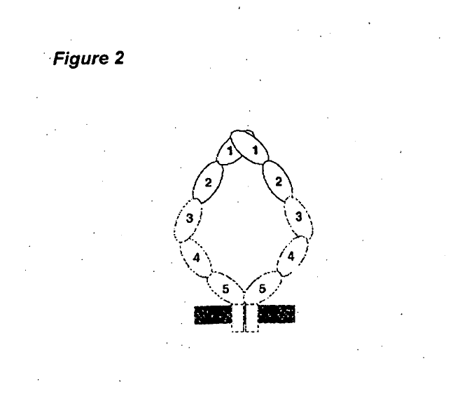

FIG. 2: Structure of ICAM-1 (intercellular adhesion molecule-1).

### EXAMPLES

### Example 1

### Experimental Data

### ICAM-1 and the BI-AB Epitope are Strongly Expressed in Multiple-Myeloma-Related Disorders

We evaluated expression of the BI-AB epitope on bone marrow cells in patients with multiple-myeloma-related disorders (plasmacytoma, plasma cell leukemia, and light chain amyloidosis) by flow cytometry (FIG. 1).

Cells from multiple-myeloma-related disorders were identified using fluorescent antibodies against the following surface antigens CD38, CD138, CD45 and CD56 according to European Myeloma Network guidelines on multiparametric flow cytometry in MM (Rawstron et al., 2008) and confirming monoclonal cells from multiple-myeloma-related disorders with intracellular staining of lambda and kappa light chains.

All patients with multiple-myeloma-related disorders expressed the BI-AB epitope on most cells from multiple-myeloma-related disorders (FIG. 1). The BI-AB epitope was generally very highly expressed on these cells, with median expression level 10 times higher than on normal B cells from the same patients. We conclude that ICAM-1 is strongly expressed on the surface of plasma cells from multiple-myeloma-related disorders.

The BI-AB antibody has been described, previously, for example in WO 2007/112110, and comprises the heavy chain variable region and light chain variable region amino acid sequences of SEQ ID NO:7 and 8, respectively, as defined herein (i.e. which comprise the CDR amino acid sequences of SEQ ID NO:1-6 defined herein).

### Example 2

### Exemplary Pharmaceutical Formulations

Whilst it is possible for an antibody of the invention to be administered alone, it is preferable to present it as a medicament or pharmaceutical formulation, together with one or more acceptable carriers. The carrier(s) must be “acceptable” in the sense of being compatible with the agent of the invention and not deleterious to the recipients thereof. Typically, the carriers will be water or saline which will be sterile and pyrogen-free.

The following examples illustrate medicaments and pharmaceutical compositions according to the invention in which the active ingredient is an antibody of the invention.

Preferably, an antibody of the invention is provided in an amount from 0.1 μg to 1 g for each administration. It will be appreciated that the following exemplary medicaments and pharmaceutical compositions may be prepared containing an amount of the antibody from 0.1 μg to 1 g. For example, the antibody may be present in a 10^{th} or 100^{th} or 200^{th} or 500^{th} of the amount shown in the following exemplary medicaments and pharmaceutical compositions with the amounts of the remaining ingredients changed accordingly.

### Example A

### Injectable Formulation

| | | |
|---|---|---|
| Active ingredient | 1 | mg |
| Sterile, pyrogen free phosphate buffer (pH 7.0) to | 10 | ml |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### Example B

### Intramuscular Injection

| | | |
|---|---|---|
| Active ingredient | 1 | mg |
| Benzyl Alcohol | 0.10 | g |
| Glucofurol 75 ® | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### Example 3

### Treatment of a Multiple-Myeloma-Related Disorder in an Individual Using an Antibody, Medicament or Pharmaceutical Composition of the Invention

An individual presenting with a multiple-myeloma-related disorder will be selected for treatment using a medicament of the invention comprising an antibody as defined herein, preferably the exemplary antibody, BI-AB.

It will be preferred that the medicament is selected from those defined in the accompanying Examples.

Administration of the medicament of the invention will be performed by the parenteral administration route at a dosage of between about 0.02 mg/ml to 5 mg/ml of the antibody.

Administration will be repeated at a frequency of between: once in every three weeks, to every other day, until the symptoms of the multiple-myeloma-related disorder are alleviated as will be recognised by a physician skilled in the art.

1. An antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
for use in the treatment of a multiple-myeloma-related disorder, the treatment comprising the step of administering to a patient in need thereof an effective amount of the antibody, antigen-binding fragment, variant, fusion or derivative thereof to treat the multiple-myeloma-related disorder.

2. Use of an antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
in the manufacture of a medicament for the treatment of a multiple-myeloma-related disorder, the treatment comprising the step of administering to a patient in need thereof an effective amount of the antibody, antigen-binding fragment, variant, fusion or derivative thereof to treat the multiple-myeloma-related disorder.

3. A method for treating a multiple-myeloma-related disorder in a patient, the method comprising the step of administering to a patient in need thereof an effective amount of:
an antibody or an antigen-binding fragment thereof with binding specificity for ICAM-1,
or a variant, fusion or derivative of said antibody or an antigen-binding fragment, or a fusion of a said variant or derivative thereof, with binding specificity for ICAM-1,
wherein the amount of the antibody, antigen-binding fragment, variant, fusion or derivative thereof is effective to treat the multiple-myeloma-related disorder.

4. The antibody, use or method according to claims 1-3 wherein the patient having the multiple-myeloma-related disorder does not additionally have multiple-myeloma.

5. The antibody, use or method according to claims 1-4 wherein the multiple-myeloma-related disorder is selected from the group comprising: Plasmacytoma (PC); Plasma Cell Leukemia (PCL); Light Chain Amyloidosis (AL).

6. The antibody, use or method according to any one of the preceding claims wherein the effective amount of the antibody, antigen-binding fragment, variant, fusion or derivative thereof is between about 0.1 μg to 1 g of the antibody (for example between about 0.02 mg/ml to 5 mg/ml), antigen-binding fragment, variant, fusion or derivative thereof.

7. The antibody, use or method according to any one of the preceding claims wherein ICAM-1 is localised on the surface of, a plasma cell.

8. The antibody, use or method according to any one of the preceding claims wherein the antibody or antigen-binding fragment, or variant, fusion or derivative thereof, is capable of specifically binding ICAM-1 localised on the surface of a cell and inhibiting and/or preventing proliferation of that cell.

9. The antibody, use or method according to any one of the preceding claims wherein the antibody or antigen-binding fragment, or variant, fusion or derivative thereof, is capable of specifically binding ICAM-1 localised on the surface of a cell and inducing apoptosis of that cell.

10. The antibody, use or method according to any one of the preceding claims wherein the antibody or antigen-binding fragment, or variant, fusion or derivative thereof, is capable of specifically binding ICAM-1 localised on the surface of a cell and inducing antibody-dependent cell cytotoxicity against that cell.

11. The antibody, use or method according to any one of the preceding claims wherein the antibody or antigen-binding fragment has efficacy in the treatment of a multiple-myeloma-related disorder.

12. The antibody, use or method according to claim 11 wherein the multiple-myeloma-related disorder is Plasmacytoma (PC).

13. The antibody, use or method according to claim 11 wherein the multiple-myeloma-related disorder is Plasma Cell Leukemia (PCL).

14. The antibody, use or method according to claim 11 wherein the multiple-myeloma-related disorder is Light Chain Amyloidosis (AL).

15. The antibody, use or method according to any preceding claim wherein the antibody or antigen-binding fragment, or a variant, fusion or derivative thereof, comprises or consists of an intact antibody.

16. The antibody, use or method according to any one of the preceding claims wherein the antibody or antigen-binding fragment, or a variant, fusion or derivative thereof, comprises or consists of an antigen-binding fragment selected from the group consisting of: an Fv fragment; an Fab fragment; an Fab-like fragment.

17. The antibody, use or method according to claim 16 wherein the Fv fragment is a single chain Fv fragment or a disulphide-bonded Fv fragment.

18. The antibody, use or method according to claim 17 wherein the Fab-like fragment is an Fab′ fragment or an F(ab)₂ fragment.

19. The antibody, use or method according to any one of the preceding claims wherein the antibody is a recombinant antibody.

20. The antibody, use or method according to any one of the preceding claims wherein the antibody is a monoclonal antibody.

21. The antibody, use or method according to any one of the preceding claims wherein the antibody or antigen-binding fragment thereof is a human antibody or humanised antibody.

22. The antibody, use or method according to any one of the preceding claims wherein the antibody or antigen-binding fragment thereof comprises one or more of the following amino acid sequences:
[SEQ ID NO: 1]
FSNAWMSWVRQAPG;
and/or
[SEQ ID NO: 2]
AFIWYDGSNKYYADSVKGR;
and/or
[SEQ ID NO: 3]
ARYSGWYFDY;
and/or
[SEQ ID NO: 4]
CTGSSSNIGAGYDVH;
and/or
[SEQ ID NO: 5]
DNNNRPS;
and/or
[SEQ ID NO: 6]
CQSYDSSLSAWL.

23. The antibody, use or method according to claim 22 wherein the heavy chain variable region of the antibody, fragment, variant, fusion or derivative comprises the following CDRs:
[SEQ ID NO: 1]
FSNAWMSWVRQAPG;
and
[SEQ ID NO: 2]
AFIWYDGSNKYYADSVKGR;
and
[SEQ ID NO: 3]
ARYSGWYFDY.

24. The antibody, use or method according to claim 23 wherein the heavy chain variable region of the antibody, fragment, variant, fusion or derivative comprises the amino acid sequence of SEQ ID NO: 7.
[SEQ ID NO: 7]
EVQLLESGGGLVQPGGSLRLSCAASGFTFSNAWMSWVRQAPGKGLEWVAF
IWYDGSNKYYADSVKGRFTISRDNSKNTLYLQMNSLRAEDTAVYYCARYS
GWYFDYWGQGTLVTVSS

25. The antibody, use or method according to claim 22 wherein the light chain variable region of the antibody, fragment, variant, fusion or derivative comprises the following CDRs:
[SEQ ID NO: 4]
CTGSSSNIGAGYDVH;
and
[SEQ ID NO: 5]
DNNNRPS;
and
[SEQ ID NO: 6]
CQSYDSSLSAWL.

26. The antibody, use or method according to claim 25 wherein the light chain variable region of the antibody, fragment, variant, fusion or derivative comprises the amino acid sequence of SEQ ID NO: 8.
[SEQ ID NO: 8]
QSVLTQPPSASGTPGQRVTISCTGSSSNIGAGYDVHWYQQLPGTAPKLLI
YDNNNRPSGVPDRFSGSKSGTSASLAISGLRSEDEADYYCQSYDSSLSAW
LFGGGTKLTVLG

27. The antibody, use or method according to any one of the preceding claims wherein the antibody or antigen-binding fragment, variant, fusion or derivative comprises a heavy chain variable region as defined in claim 23 or 24 and a light chain variable region as defined in claim 25 or 26.

28. The antibody, use or method according to any one of the preceding claims wherein the antibody or antigen-binding fragment, variant, fusion or derivative comprises a heavy chain variable region as defined in claim 24 and a light chain variable region as defined in claim 26.

29. The antibody, use or method according to any one of the preceding claims wherein the antibody or antigen-binding fragment, variant, fusion or derivative is capable of competing for binding to ICAM-1 with an antibody as defined in claim 27 or claim 28.

30. An antibody or antigen-binding fragment thereof for use in treating a multiple-myeloma-related disorder substantially as described herein with reference to the description.

31. Use of an antibody or antigen-binding fragment thereof substantially as described herein with reference to the description.

32. A method of treating a multiple-myeloma-related disorder in an individual substantially as described herein.
